(19) United States
(12) Patent Application Publication
(10) Pub. No.: US 2022/0273324 A1
(43) Pub. Date: Sep. 1, 2022

(54) VALVULOPLASTY TREATMENT ASSEMBLY AND METHOD USING DIRECTED BUBBLE ENERGY

(71) Applicant: Bolt Medical, Inc., Carlsbad, CA (US)

(72) Inventor: Eric Schultheis, San Clemente, CA (US)

(21) Appl. No.: 17/677,834

(22) Filed: Feb. 22, 2022

Related U.S. Application Data
(60) Provisional application No. 63/154,982, filed on Mar. 1, 2021.

(51) Int. Cl. A61B 17/22; A61B 18/26

(52) U.S. Cl. CPC A61B 17/22022; A61B 18/26; A61B 2018/266; A61B 2017/22008; A61B 2017/22098

(57) ABSTRACT

A catheter system for treating a treatment site within or adjacent to the heart valve within a body of a patient includes an energy source, an energy guide and an energy director. The energy source generates energy. The energy guide includes a guide proximal end and a guide distal end. The energy guide is configured to receive energy from the energy source and guide the energy from the guide proximal end toward the guide distal end. The energy director includes a director wall that defines a director interior, and a director distal end that is selectively positioned substantially adjacent to the treatment site. The guide distal end of the energy guide is positioned within the director interior. The director distal end is at least partially open toward the treatment site.

### RELATED APPLICATION

This application claims priority from U.S. Provisional Application Ser. No. 63/154,982, filed on Mar. 1, 2021. To the extent permitted, the contents of U.S. Provisional Application Ser. No. 63/154,982 are incorporated in their entirety herein by reference.

### BACKGROUND

Vascular lesions, such as calcium deposits, within and adjacent to heart valves in the body can be associated with an increased risk for major adverse events, such as myocardial infarction, embolism, deep vein thrombosis, stroke, and the like. Severe vascular lesions, such as severely calcified vascular lesions, can be difficult to treat and achieve patency for a physician in a clinical setting.

The aortic valve is a valve of the human heart between the left ventricle and the aorta. The aortic valve functions as a one-way valve and typically includes three leaflets which open and close in unison when the valve is functioning properly. During normal operation, when the left ventricle contracts (during ventricular systole), pressure rises in the left ventricle. When the pressure in the left ventricle rises above the pressure in the aorta, the aortic valve opens, allowing blood to exit the left ventricle into the aorta. When ventricular systole ends, pressure in the left ventricle rapidly drops. When the pressure in the left ventricle decreases, the momentum of the vortex at the outlet of the valve forces the aortic valve to close. Dysfunction or improper operation of the aortic valve can result in left ventricular hypertrophy (enlargement and thickening of the walls of the left ventricle) and/or aortic valve regurgitation, which is the backflow of blood from the aorta into the left ventricle during diastole. Such issues can lead to heart failure if left uncorrected.

A calcium deposit on the aortic valve, known as aortic valve stenosis, can form adjacent to a valve wall of the aortic valve and/or on or between the leaflets of the aortic valve. Aortic valve stenosis can prevent the leaflets from opening and closing completely, which can, in turn, result in the undesired aortic valve regurgitation. Over time, such calcium deposits can cause the leaflets to become less mobile and ultimately prevent the heart from supplying enough blood to the rest of the body.

Certain methods are currently available which attempt to address aortic valve stenosis, but such methods have not been altogether satisfactory. Certain such methods include using a standard balloon valvuloplasty catheter, and artificial aortic valve replacement, which can be used to restore functionality of the aortic valve. During aortic valvuloplasty, a balloon is expanded at high pressure in the inside of the aortic valve to break apart calcification on the valve leaflets cusps and between the commissures of the valve leaflets. Usually, this procedure is done prior to placing a replacement aortic valve. Certain anatomical factors such as heavily calcified valves can prevent the valvuloplasty from being effective enough for valve placement, causing performance and safety concerns for the replacement valve. In order for the replacement valve to function correctly it must be precisely positioned over the native valve. Stated in another manner, aortic valvuloplasty often does not have enough strength to sufficiently disrupt the calcium deposit between the leaflets or at the base of the leaflets, which can subsequently adversely impact the effectiveness of any aortic valve replacement procedure. Aortic valve replacement can also be highly invasive and extremely expensive. In still another such method, a valvular stent can be placed between the leaflets to bypass the leaflets. This procedure is relatively costly, and results have found that the pressure gradient does not appreciably improve.

Thus, there is an ongoing desire to develop improved methodologies for valvuloplasty in order to more effectively and efficiently break up calcium deposits adjacent to the valve wall of the aortic valve and/or on or between the leaflets of the aortic valve. Additionally, it is desired that such improved methodologies work effectively to address not only aortic valve stenosis related to the aortic valve, but also calcification on other heart valves, such as mitral valve stenosis within the mitral valve, valvular stenosis within the tricuspid valve, and pulmonary valve stenosis within the pulmonary valve.

### SUMMARY

The present invention is directed toward a catheter system for placement within a heart valve. The catheter system can be used for treating a treatment site within or adjacent to the heart valve within a body of a patient. In various embodiments, the catheter system includes an energy source, an energy guide and an energy director. The energy source generates energy. The energy guide includes a guide proximal end and a guide distal end. The energy guide is configured to receive energy from the energy source and guide the energy from the guide proximal end toward the guide distal end. The energy director includes a director wall that defines a director interior, and a director distal end that is selectively positioned substantially adjacent to the treatment site. The guide distal end of the energy guide is positioned within the director interior. The director distal end is at least partially open toward the treatment site.

In some embodiments, the energy director is substantially cone-shaped, the energy director further including a director proximal end that is smaller than the director distal end.

In certain embodiments, the director distal end is fully open toward the treatment site. In other embodiments, the guide distal end is partially closed and includes a director aperture that is open in a direction toward the treatment site.

In some embodiments, the energy director is substantially spherical-shaped. In such embodiments, the guide distal end can be partially closed and can include a director aperture that is open in a direction toward the treatment site.

In certain embodiments, the energy director is configured to receive a catheter fluid within the director interior.

In some embodiments, the energy guide guides the energy into the catheter fluid within the director interior so that plasma is formed in the catheter fluid within the director interior.

In certain embodiments, the plasma formation causes rapid bubble formation and generates one or more pressure waves within the catheter fluid that impart a force upon the treatment site.

In various embodiments, the energy source generates pulses of energy that are guided along the energy guide into the catheter fluid within the director interior so that the plasma is formed in the catheter fluid within the director interior.

In some embodiments, the catheter system further includes a second energy guide including a second guide proximal end and a second guide distal end, the second energy guide being configured to receive energy from the energy source and guide the energy from the second guide proximal end toward the second guide distal end.

In various embodiments, the second guide distal end of the second energy guide is positioned within the director interior.

In certain embodiments, the catheter system further includes an assembly shaft, wherein the energy director is coupled to the assembly shaft.

In some embodiments, the assembly shaft includes an inflation port, and a catheter fluid is directed into the director interior of the energy director through the inflation port.

In certain embodiments, the assembly shaft includes an energy guide lumen, and at least a portion of the energy guide extends through the energy guide lumen. In some embodiments, the assembly shaft is substantially cylindrical-shaped.

In some embodiments, the guide distal end of the energy guide is selectively steerable within the director interior.

In various embodiments, the catheter system further includes a steering member that is coupled to the energy guide so that the guide distal end is selectively steerable within the director interior.

In various embodiments, the energy director is selectively movable between a retracted position and a deployed position.

In certain embodiments, when the energy director is in the retracted position, the energy director is positioned substantially within an outer sheath.

In some embodiments, when the energy director is in the deployed position, the energy director is positioned outside of and extends away from the outer sheath.

In certain embodiments, when the energy director is in the deployed position, the director distal end is positioned substantially adjacent to the treatment site.

In some such embodiments, when the energy director is in the retracted position, the energy director is configured to be in a collapsed state.

In various embodiments, when the energy director is in the deployed position, the energy director can be configured to move from the collapsed state to an expanded state.

In certain embodiments, the catheter system further includes an expansion assistance structure that is coupled to the director wall, the expansion assistance structure being configured to assist the energy director to move from the collapsed state to the expanded state.

In some embodiments, the expansion assistance structure is self-expanding.

In various embodiments, the expansion assistance structure can be a lattice-like structure.

In certain embodiments, the expansion assistance structure can be formed from one or more of metallic materials, nitinol and plastic.

In certain embodiments, wherein the heart valve includes one or more leaflets, the catheter system further includes a leaflet support assembly including a support shaft that is configured to extend through the heart valve, and a leaflet supporter that is coupled to the support shaft and extends substantially perpendicularly away from the support shaft.

In some embodiments, the director distal end of the energy director and the leaflet supporter are selectively positionable on opposite sides of one of the leaflets.

In various embodiments, the catheter system further includes a second energy director including a second director wall that defines a second director interior, and a second director distal end that is selectively positioned substantially adjacent to the treatment site.

In some embodiments, each of the energy director and the second energy director are selectively positionable within a common outer sheath.

In various embodiments, the energy director is selectively positionable within a first outer sheath, and the second energy director is selectively positionable within a second outer sheath that is different than the first outer sheath.

In certain embodiments, the catheter system can further include a second energy guide including a second guide proximal end and a second guide distal end, the second energy guide being configured to receive energy from the energy source and guide the energy from the second guide proximal end to the second guide distal end.

In various embodiments, the second guide distal end of the second energy guide is positioned within the second director interior.

In certain embodiments, the energy director is formed from one or more of silicone, plastic, spun polytetrafluoroethylene (PTFE), nylon, and fibers such as polyester fiber and nylon fiber.

In some embodiments, the energy source is a laser source that provides pulses of laser energy.

In certain embodiments, the energy guide includes an optical fiber.

In some embodiments, the energy source is a high voltage energy source that provides pulses of high voltage.

In various embodiments, the energy guide can include an electrode pair including spaced apart electrodes that extend into the director interior, and pulses of high voltage from the energy source can be applied to the electrodes and form an electrical arc across the electrodes.

The present invention is also directed toward a method for treating a treatment site within or adjacent to a heart valve within a body of a patient, the method including the steps of generating energy with an energy source; receiving energy from the energy source with an energy guide; guiding the energy from the energy source with the energy guide from a guide proximal end to a guide distal end; positioning a director distal end of an energy director substantially adjacent to the treatment site, the director distal end being at least partially open toward the treatment site, the energy director including a director wall that defines a director interior; and positioning the guide distal end of the energy guide within the director interior.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic cross-sectional view of an embodiment of a catheter system in accordance with various embodiments herein, the catheter system including a valvular lithotripsy treatment assembly having features of the present invention;

FIG. 2 is a simplified schematic view of a portion of a heart valve and a portion of an embodiment of the valvular lithotripsy treatment assembly;

FIG. 3 is a simplified schematic view of a portion of the heart valve and a portion of another embodiment of the valvular lithotripsy treatment assembly;

FIG. 4 is a simplified schematic view of a portion of the heart valve and a portion of still another embodiment of the valvular lithotripsy treatment assembly;

FIG. 5 is a simplified schematic view of a portion of the heart valve and a portion of another embodiment of the valvular lithotripsy treatment assembly;

FIG. 6 is a simplified schematic view of a portion of the heart valve and a portion of yet another embodiment of the valvular lithotripsy treatment assembly;

FIG. 7 is a simplified schematic view of a portion of the heart valve and a portion of another embodiment of the valvular lithotripsy treatment assembly;

FIG. 8 is a simplified schematic view of a portion of the heart valve and a portion of still another embodiment of the valvular lithotripsy treatment assembly;

FIG. 9 is a simplified schematic view of a portion of the heart valve and a portion of yet another embodiment of the valvular lithotripsy treatment assembly; and

FIG. 10 is a simplified schematic view of a portion of the heart valve and a portion of still yet another embodiment of the valvular lithotripsy treatment assembly.

While embodiments of the present invention are susceptible to various modifications and alternative forms, specifics thereof have been shown by way of example and drawings, and are described in detail herein. It is understood, however, that the scope herein is not limited to the particular embodiments described. On the contrary, the intention is to cover modifications, equivalents, and alternatives falling within the spirit and scope herein.

### DESCRIPTION

Treatment of vascular lesions can reduce major adverse events or death in affected subjects. As referred to herein, a major adverse event is one that can occur anywhere within the body due to the presence of a vascular lesion. Major adverse events can include, but are not limited to, major adverse cardiac events, major adverse events in the peripheral or central vasculature, major adverse events in the brain, major adverse events in the musculature, or major adverse events in any of the internal organs.

The catheter systems and related methods disclosed herein are configured to incorporate improved methodologies for valvular lithotripsy in order to more effectively and efficiently break up any vascular lesions that may have developed on and/or within the heart valves over time. In particular, the catheter systems and related methods generally include a catheter including a valvular lithotripsy treatment assembly (also sometimes referred to herein simply as a “treatment assembly”) that is configured to advance to a vascular lesion, such as a calcified vascular lesion or a fibrous vascular lesion, at a treatment site located within or adjacent a heart valve within a body of a patient. More specifically, at least a portion of the treatment assembly is selectively positioned adjacent to the valve wall and/or on or between adjacent leaflets within the heart valve in order to break up the vascular lesions. While such methodologies are often described herein as being useful for treatment of aortic valve stenosis in relation to the aortic valve, it is appreciated that such methodologies are also useful in treatment of calcium deposits on other heart valves, such as for mitral valve stenosis within the mitral valve, for valvular stenosis within the tricuspid valve, and/or for pulmonary valve stenosis within the pulmonary valve.

In certain embodiments shown and described herein, the catheter systems and related methods utilize an energy source, e.g., a light source such as a laser source or another suitable energy source, which provides energy that is guided by one or more energy guides, e.g., light guides such as optical fibers, which are disposed along a catheter shaft and within the director interior of the energy director to create a localized plasma in the catheter fluid that is retained within the director interior of the energy director. As such, the energy guides can be said to incorporate a “plasma generator” at or near a guide distal end of the energy guide that is positioned within the director interior of the energy director located at the treatment site. The creation of the localized plasma can initiate the rapid formation of one or more bubbles that can rapidly expand to a maximum size and then dissipate through a cavitation event that can launch a pressure wave upon collapse. In particular, the rapid expansion of the plasma-induced bubbles (also sometimes referred to simply as “plasma bubbles”) can generate one or more pressure waves within the catheter fluid retained within the director interior of the energy director.

As used herein, the terms “treatment site”, “intravascular lesion” and “vascular lesion” are used interchangeably unless otherwise noted. Also, the intravascular lesions and/or the vascular lesions are sometimes referred to herein simply as “lesions”.

Those of ordinary skill in the art will realize that the following detailed description of the present invention is illustrative only and is not intended to be in any way limiting. Other embodiments of the present invention will readily suggest themselves to such skilled persons having the benefit of this disclosure. Additionally, other methods of delivering energy to the lesions can be utilized, including, but not limited to electric current-induced plasma generation. Reference will now be made in detail to implementations of the present invention as illustrated in the accompanying drawings. The same or similar nomenclature and/or reference indicators will be used throughout the drawings and the following detailed description to refer to the same or like parts.

In the interest of clarity, not all of the routine features of the implementations described herein are shown and described. It is appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it is recognized that such a development effort might be complex and time-consuming, but would nevertheless be a routine undertaking of engineering for those of ordinary skill in the art having the benefit of this disclosure.

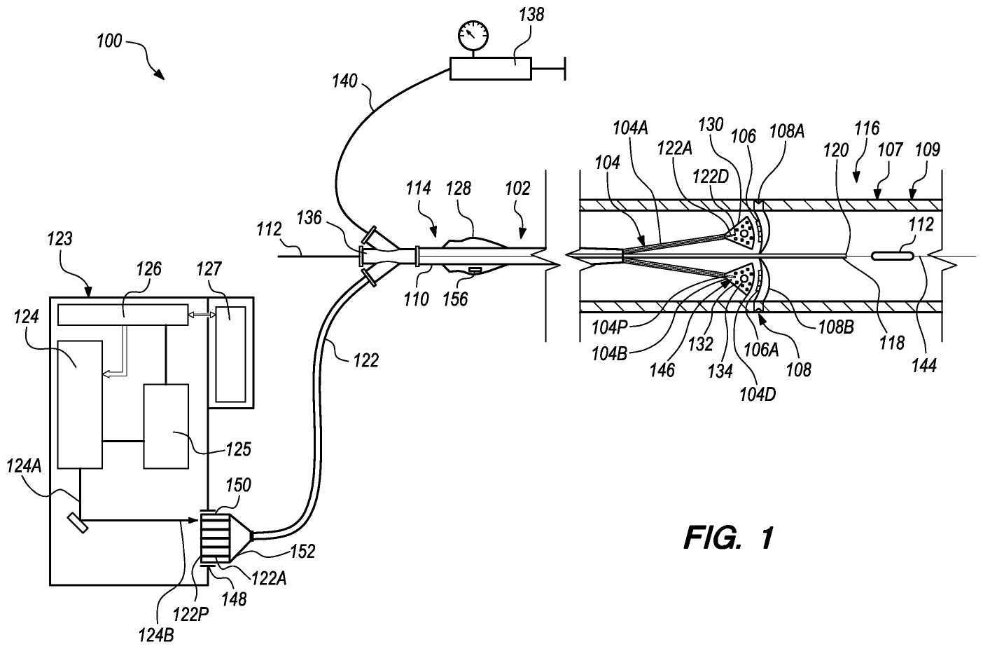

The catheter systems disclosed herein can include many different forms. Referring now to FIG. 1, a schematic cross-sectional view is shown of a catheter system 100 in accordance with various embodiments. The catheter system 100 is suitable for imparting pressure waves to induce fractures in one or more treatment sites within or adjacent leaflets within the aortic valve or another appropriate heart valve. In the embodiment illustrated in FIG. 1, the catheter system 100 can include one or more of a catheter 102, an energy guide bundle 122 including one or more energy guides 122A, a source manifold 136, a fluid pump 138, a system console 123 including one or more of an energy source 124, a power source 125, a system controller 126, and a graphic user interface 127 (a “GUI”), and a handle assembly 128. Additionally, as described herein, the catheter 102 includes a valvular lithotripsy treatment assembly 104 (also sometimes referred to herein simply as a “treatment assembly”) that is configured to be selectively positioned adjacent to a valve wall 108A (including annulus and commissures) and/or on or between adjacent leaflets 108B within a heart valve 108, e.g., the aortic valve, at a treatment site 106. Alternatively, the catheter system 100 can have more components or fewer components than those specifically illustrated and described in relation to FIG. 1.

The catheter 102 is configured to move to a treatment site 106 within or adjacent to the heart valve 108 within a body 107 of a patient 109. The treatment site 106 can include one or more vascular lesions 106A such as calcified vascular lesions, for example. Additionally, or in the alternative, the treatment site 106 can include vascular lesions 106A such as fibrous vascular lesions.

As illustrated in this embodiment, the catheter 102 can include a catheter shaft 110, a guide shaft 118, the treatment assembly 104, and a guidewire 112.

The catheter shaft 110 can extend from a proximal portion 114 of the catheter system 100 to a distal portion 116 of the catheter system 100. The catheter shaft 110 can include a longitudinal axis 144. The guide shaft 118 can be positioned, at least in part, within the catheter shaft 110. The guide shaft 118 can define a guidewire lumen which is configured to move over the guidewire 112 and/or through which the guidewire 112 extends. The catheter shaft 110 can further include one or more inflation lumens (not shown in FIG. 1) and/or various other lumens for various other purposes. In some embodiments, the catheter 102 can have a distal end opening 120 and can accommodate and be tracked over the guidewire 112 as the catheter 102 is moved and positioned at or near the treatment site 106.

The treatment assembly 104 is coupled to the catheter shaft 110. The design of the treatment assembly 104 can be varied. In the embodiment shown in FIG. 1, the treatment assembly 104 includes an assembly shaft 104A, and an energy director 104B that is coupled and/or secured to the assembly shaft 104A. In some embodiments, the treatment assembly 104 and/or the energy director 1046 is movable between a retracted position and a deployed position. When in the retracted position, the treatment assembly 104 and/or the energy director 104B can be positioned substantially within an outer sheath (not shown in FIG. 1), such as the catheter shaft 110 or another suitable shaft or sheath, so as to better enable the treatment assembly 104 and/or the catheter 102 to advance through a patient's vasculature. When in the deployed position, the treatment assembly 104 and/or the energy director 104B is positioned outside of and/or extending away from the outer sheath, such as the catheter shaft 110 or other suitable shaft or sheath, so that the energy director 1046 of the treatment assembly 104 can be positioned substantially adjacent to the vascular lesions 106A at the treatment site 106. It is appreciated that a director proximal end 104P of the energy director 104B can be positioned substantially adjacent to the assembly shaft 104A, and a director distal end 104D of the energy director 104B can be positioned substantially directly adjacent to and/or abutting vascular lesions 106A at the treatment site 106, when the treatment assembly 104 is in the deployed position. Additionally, when the treatment assembly 104 and/or the energy director 104B is moved to the deployed position, the energy director 1046 is allowed to expand into a desired shape, such as being substantially cone-shaped and/or substantially spherical-shaped in certain non-exclusive embodiments. Stated in another manner, when the treatment assembly 104 and/or the energy director 104B is moved to the deployed position, the energy director 1046 can also move from a collapsed state to an expanded state. In some embodiments, the energy director 1046 is configured to self-expand to its natural expanded state when moved to the deployed position. Alternatively, in other embodiments, the energy director 104B can include and/or incorporate an expanding lattice structure, such as an expanding metallic lattice structure, so as to help the energy director 104B expand to its desired expanded state when the energy director 1046 is moved to the deployed position.

It is appreciated that in certain alternative embodiments, the treatment assembly 104 can include a plurality of assembly shafts and a plurality of energy directors, with one energy director being coupled and/or secured to each of the assembly shafts. In different such embodiments, each of the assembly shafts and corresponding energy directors can have substantially the same design, or one or more of the assembly shafts and/or the energy directors can have different designs from one another. Stated in another manner, in such embodiments, the catheter system 100 can be said to include a plurality of treatment assemblies 104, each treatment assembly 104 including an assembly shaft 104A and an energy director 1046 that is coupled and/or secured to the assembly shaft 104A. With such design, the treatment assemblies 104 are able to treat vascular lesions 106A on multiple leaflets 108B of the heart valve 108 substantially contemporaneously. Additionally, in certain such embodiments, at least two of the plurality of treatment assemblies 104 can be positioned at least in part within a common outer sheath. Alternatively, in other such embodiments, each treatment assembly 104 can be positioned within its own outer sheath.

The assembly shaft 104A can define an inflation and/or irrigation lumen through which a catheter fluid 132 can be transmitted to the energy director 104B. Additionally, the assembly shaft 104A can further define one or more energy guide lumens through which the one or more energy guides 122A can extend. In some embodiments, the assembly shaft 104A can be substantially cylindrical tube-shaped. Alternatively, the assembly shaft 104A can have another suitable shape.

In certain embodiments, as shown, the energy director 104B has a director wall 130 that defines a director interior 146. During use of the treatment assembly 104, the energy director 104B is configured to receive and retain the catheter fluid 132 substantially within the director interior 146 of the energy director 104B. In various embodiments, as described in greater detail herein below, the retaining of the catheter fluid 132 within the director interior 146 of the energy director 1046 enables the creation of a plasma within the director interior 146, and the energy director 1046 is configured to direct the energy from the plasma, such as in the form of one or more plasma bubbles 134 and/or corresponding pressure waves, toward the vascular lesions 106A at the treatment site 106. Additionally, the catheter fluid 132 being directed into and retained within the director interior 146 of the energy director 104B is also utilized to inhibit blood from entering into the director interior 146 so as to reduce the risk of blood coagulation.

In various embodiments, the director distal end 104D of the energy director 104B can be open toward the vascular lesions 106A at the treatment site 106 so that the energy director 104B can effectively direct energy through the open director distal end 104D and toward the vascular lesions 106A. In some embodiments, the energy director 104B can further include an aperture (not shown in FIG. 1) on a face of the energy director 104B at the director distal end 104D to help maintain a desired fluid pressure within the director interior 146 so as to better maintain the desired expanded shape, such as the conical shape, when in the deployed position. The aperture can also help to better and more precisely direct the bubble energy from the plasma bubbles 134 toward the vascular lesions 106A at the treatment site 106.

The energy director 104B can be formed from any suitable natural and/or synthetic materials. In some embodiments, the energy director 104B can be designed to include either compliant, semi-compliant or non-compliant materials that will allow the energy director 104B to fold into the outer sheath during movement to the retracted position. For example, in certain non-exclusive alternative embodiments, the energy director 104B can be formed from one or more of silicone, plastic, polydimethylsiloxane (PDMS), polyurethane, polymers such as PEBAX™ material, nylon, Gortex®, Mylar®, spun polytetrafluoroethylene (PTFE), fibers such as polyester fiber and nylon fiber, or any other suitable material.

The energy director 104B can have various shapes when in the deployed position and in the expanded state. For example, in some embodiments, the energy director 104B can be substantially cone-shaped, with a narrow, circular-shaped, director proximal end 104P and a wider, circular-shaped, director distal end 104D, when in the deployed position and in the expanded state. Alternatively, in other embodiments, the energy director 104B can be substantially spherical-shaped when in the deployed position and in the expanded state. Still alternatively, the energy director 104B can have another suitable shape when in the deployed position and in the expanded state.

In certain embodiments, the director distal end 104D can be at least partially open toward the vascular lesions 106A when the energy director 104B is in the deployed position. With such design, the energy director 104B is better able direct the energy as desired toward the vascular lesions 106A at the treatment site 106. For example, in one such embodiment, the director distal end 104D can be fully open so that the energy director 104B is able to direct and/or guide energy through the open director distal end 104D toward the vascular lesions 106A at the treatment site 106. Alternatively, in another such embodiment, the director distal end 104D can be partially closed with an aperture formed thereon. The aperture formed in the director distal end 104D can enable a more targeted directing and/or guiding of the energy toward the vascular lesions 106A. Additionally, the aperture can also help maintain a desired fluid pressure within the director interior 146 so as to better maintain the desired expanded shape, such as the conical shape, when in the deployed position.

The director distal end 104D of the energy director 104B can have any suitable diameter when in the deployed position and in the expanded state. In various embodiments, the director distal end 104D of the energy director 104B can have a diameter when in the deployed position and in the expanded state ranging from less than one millimeter (mm) up to 25 mm. Alternatively, the director distal end 104D of the energy director 1046 can have a diameter that is larger or smaller than the noted range of values when in the deployed position and in the expanded state.

In some embodiments, the energy director 104B can have a length from the director proximal end 104P to the director distal end 104D ranging from at least one mm to 50 mm. Alternatively, the energy director 104B can have a length that is different than the noted range of values.

The catheter fluid 132 can be a liquid or a gas. Some examples of the catheter fluid 132 suitable for use can include, but are not limited to one or more of water, saline, contrast medium, fluorocarbons, perfluorocarbons, gases, such as carbon dioxide, or any other suitable catheter fluid 132. In some embodiments, the catheter fluid 132 can be used as a base inflation fluid. In some embodiments, the catheter fluid 132 can include a mixture of saline to contrast medium in a volume ratio of approximately 50:50. In other embodiments, the catheter fluid 132 can include a mixture of saline to contrast medium in a volume ratio of approximately 25:75. In still other embodiments, the catheter fluid 132 can include a mixture of saline to contrast medium in a volume ratio of approximately 75:25. However, it is understood that any suitable ratio of saline to contrast medium can be used. The catheter fluid 132 can be tailored on the basis of composition, viscosity, and the like so that the rate of travel of the pressure waves are appropriately manipulated. In certain embodiments, the catheter fluids 132 suitable for use are biocompatible. A volume of catheter fluid 132 can be tailored by the chosen energy source 124 and the type of catheter fluid 132 used.

In some embodiments, contrast agents used in the contrast media can include, but are not to be limited to, iodine-based contrast agents, such as ionic or non-ionic iodine-based contrast agents. Some non-limiting examples of ionic iodine-based contrast agents include diatrizoate, metrizoate, iothalamate, and ioxaglate. Some non-limiting examples of non-ionic iodine-based contrast agents include iopamidol, iohexol, ioxilan, iopromide, iodixanol, and ioversol. In other embodiments, non-iodine based contrast agents can be used. Suitable non-iodine containing contrast agents can include gadolinium (III)-based contrast agents. Suitable fluorocarbon and perfluorocarbon agents can include, but are not to be limited to, agents such as the perfluorocarbon dodecafluoropentane (DDFP, C5F12).

The catheter fluids 132 can include those that include absorptive agents that can selectively absorb light in the ultraviolet region (e.g., at least ten nanometers (nm) to 400 nm), the visible region (e.g., at least 400 nm to 780 nm), or the near-infrared region (e.g., at least 780 nm to 2.5 μm) of the electromagnetic spectrum. Suitable absorptive agents can include those with absorption maxima along the spectrum from at least ten nm to 2.5 μm. Alternatively, the catheter fluids 132 can include those that include absorptive agents that can selectively absorb light in the mid-infrared region (e.g., at least 2.5 μm to 15 μm), or the far-infrared region (e.g., at least 15 μm to one mm) of the electromagnetic spectrum. In various embodiments, the absorptive agent can be those that have an absorption maximum matched with the emission maximum of the laser used in the catheter system 100. By way of non-limiting examples, various lasers usable in the catheter system 100 can include neodymium:yttrium-aluminum-garnet (Nd:YAG−emission maximum=1064 nm) lasers, holmium:YAG (Ho:YAG−emission maximum=2.1 μm) lasers, or erbium:YAG (Er:YAG−emission maximum=2.94 μm) lasers. In some embodiments, the absorptive agents can be water soluble. In other embodiments, the absorptive agents are not water soluble. In some embodiments, the absorptive agents used in the catheter fluids 132 can be tailored to match the peak emission of the energy source 124. Various energy sources 124 having emission wavelengths of at least ten nanometers to one millimeter are discussed elsewhere herein.

In various embodiments, the fluid properties of the catheter fluid 132 can be selected to help initiate plasma to create a rapidly expanding bubble. In one embodiment, the catheter fluid 132 can have additives such as iron dextran, or nanoparticles may be used for laser-based energy sources to help reduce optical threshold of the catheter fluid 132 to initiate plasma at low energies. Alternatively, a conductive catheter fluid 132 may be selected for voltage-based energy sources that require conductive catheter fluid to initiate plasma generation.

The catheter shaft 110 of the catheter 102 can be coupled to the one or more energy guides 122A of the energy guide bundle 122 that are in optical communication with the energy source 124. The energy guide(s) 122A can be disposed along the catheter shaft 110 and within the treatment assembly 104. In some embodiments, each energy guide 122A can be an optical fiber and the energy source 124 can be a laser. The energy source 124 can be in optical communication with the energy guides 122A at the proximal portion 114 of the catheter system 100.

In some embodiments, the catheter shaft 110 can be coupled to multiple energy guides 122A such as a first energy guide, a second energy guide, a third energy guide, etc., which can be disposed at any suitable positions about the guide shaft 118 and/or the catheter shaft 110. For example, in certain non-exclusive embodiments, two energy guides 122A can be spaced apart by approximately 180 degrees about the circumference of the guide shaft 118 and/or the catheter shaft 110; three energy guides 122A can be spaced apart by approximately 120 degrees about the circumference of the guide shaft 118 and/or the catheter shaft 110; or four energy guides 122A can be spaced apart by approximately 90 degrees about the circumference of the guide shaft 118 and/or the catheter shaft 110. Still alternatively, multiple energy guides 122A need not be uniformly spaced apart from one another about the circumference of the guide shaft 118 and/or the catheter shaft 110. More particularly, it is further appreciated that the energy guides 122A can be disposed uniformly or non-uniformly about the guide shaft 118 and/or the catheter shaft 110 to achieve the desired effect in the desired locations.

The catheter system 100 and/or the energy guide bundle 122 can include any number of energy guides 122A in optical communication with the energy source 124 at the proximal portion 114, and with the catheter fluid 132 within the director interior 146 of the energy director 104B at the distal portion 116. For example, in some embodiments, the catheter system 100 and/or the energy guide bundle 122 can include from one energy guide 122A to greater than 30 energy guides 122A.

The energy guides 122A can have any suitable design for purposes of generating plasma and/or pressure waves in the catheter fluid 132 within the director interior 146 of the energy director 104B. Thus, the general description of the energy guides 122A as light guides is not intended to be limiting in any manner, except for as set forth in the claims appended hereto. More particularly, although the catheter systems 100 are often described with the energy source 124 as a light source and the one or more energy guides 122A as light guides, the catheter system 100 can alternatively include any suitable energy source 124 and energy guides 122A for purposes of generating the desired plasma in the catheter fluid 132 within the director interior 146 of the energy director 1046. For example, in one non-exclusive alternative embodiment, the energy source 124 can be configured to provide high voltage pulses, and each energy guide 122A can include an electrode pair including spaced apart electrodes that extend into the director interior 146. In such embodiment, each pulse of high voltage is applied to the electrodes and forms an electrical arc across the electrodes, which, in turn, generates the plasma and forms the pressure waves within the catheter fluid 132 that are utilized to provide the fracture force onto the vascular lesions 106A at the treatment site 106. Still alternatively, the energy source 124 and/or the energy guides 122A can have another suitable design and/or configuration.

In certain embodiments, the energy guides 122A can include an optical fiber or flexible light pipe. The energy guides 122A can be thin and flexible and can allow light signals to be sent with very little loss of strength. The energy guides 122A can include a core surrounded by a cladding about its circumference. In some embodiments, the core can be a cylindrical core or a partially cylindrical core. The core and cladding of the energy guides 122A can be formed from one or more materials, including but not limited to one or more types of glass, silica, or one or more polymers. The energy guides 122A may also include a protective coating, such as a polymer. It is appreciated that the index of refraction of the core will be greater than the index of refraction of the cladding.

Each energy guide 122A can guide energy along its length from a guide proximal end 122P to a guide distal end 122D having at least one optical window (not shown) that is positioned within the director interior 146 of the energy director 1046. Thus, the energy guide 122A is configured to guide energy into the director interior 146 of the energy director 104B to enable the creation of the plasma bubbles 134 and corresponding pressure waves within the director interior 146 that are directed by the energy director 1046 toward the vascular lesions 106A at the treatment site 106. Alternatively, the energy guides 122A can have another suitable design and/or the energy from the energy source 124 can be guided into the director interior 146 of the energy director 104B by another suitable method.

The energy guides 122A can assume many configurations about and/or relative to the catheter shaft 110 of the catheter 102. In some embodiments, the energy guides 122A can run parallel to the longitudinal axis 144 of the catheter shaft 110. In some embodiments, the energy guides 122A can be physically coupled to the catheter shaft 110. In other embodiments, the energy guides 122A can be disposed along a length of an outer diameter of the catheter shaft 110. In yet other embodiments, the energy guides 122A can be disposed within one or more energy guide lumens within the catheter shaft 110.

The energy guides 122A can also be disposed at any suitable positions about the circumference of the guide shaft 118 and/or the catheter shaft 110, and the guide distal end 122D of each of the energy guides 122A can be disposed at any suitable longitudinal position relative to the length of the treatment assembly 104 (the energy director 104B) and/or relative to the length of the guide shaft 118.

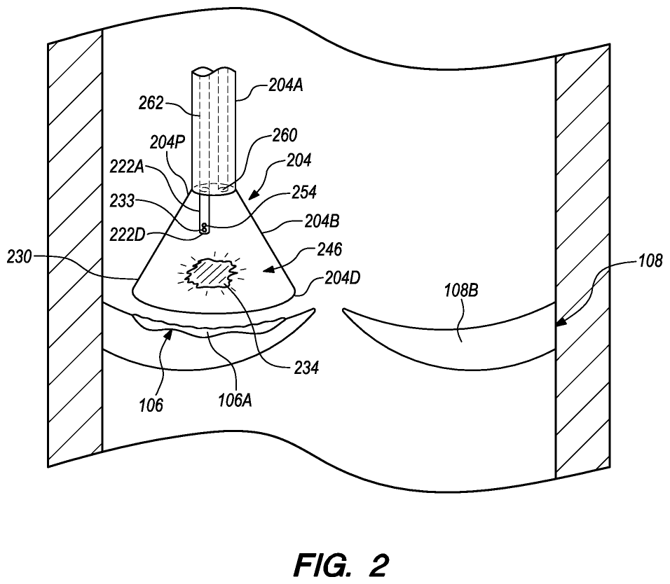

In certain embodiments, the energy guides 122A can include one or more photoacoustic transducers 254 (illustrated in FIG. 2), where each photoacoustic transducer 254 can be in optical communication with the energy guide 122A within which it is disposed. In some embodiments, the photoacoustic transducers 254 can be in optical communication with the guide distal end 122D of the energy guide 122A. Additionally, in such embodiments, the photoacoustic transducers 254 can have a shape that corresponds with and/or conforms to the guide distal end 122D of the energy guide 122A.

The photoacoustic transducer 254 is configured to convert light energy into an acoustic wave at or near the guide distal end 122D of the energy guide 122A. The direction of the acoustic wave can be tailored by changing an angle of the guide distal end 122D of the energy guide 122A.

In certain embodiments, the photoacoustic transducers 254 disposed at the guide distal end 122D of the energy guide 122A can assume the same shape as the guide distal end 122D of the energy guide 122A. For example, in certain non-exclusive embodiments, the photoacoustic transducer 254 and/or the guide distal end 122D can have a conical shape, a convex shape, a concave shape, a bulbous shape, a square shape, a stepped shape, a half-circle shape, an ovoid shape, and the like. The energy guide 122A can further include additional photoacoustic transducers 254 disposed along one or more side surfaces of the length of the energy guide 122A.

In some embodiments, the energy guides 122A can further include one or more diverting features or “diverters” (not shown in FIG. 1) within the energy guide 122A that are configured to direct energy to exit the energy guide 122A toward a side surface which can be located at or near the guide distal end 122D of the energy guide 122A, and toward the director wall 130. A diverting feature can include any feature of the system that diverts energy from the energy guide 122A away from its axial path toward a side surface of the energy guide 122A. Additionally, the energy guides 122A can each include one or more optical windows disposed along the longitudinal or circumferential surfaces of each energy guide 122A and in optical communication with a diverting feature. Stated in another manner, the diverting features can be configured to direct energy in the energy guide 122A toward a side surface that is at or near the guide distal end 122D, where the side surface is in optical communication with an optical window. The optical windows can include a portion of the energy guide 122A that allows energy to exit the energy guide 122A from within the energy guide 122A, such as a portion of the energy guide 122A lacking a cladding material on or about the energy guide 122A.

Examples of the diverting features suitable for use include a reflecting element, a refracting element, and a fiber diffuser. The diverting features suitable for focusing energy away from the tip of the energy guides 122A can include, but are not to be limited to, those having a convex surface, a gradient-index (GRIN) lens, and a mirror focus lens. Upon contact with the diverting feature, the energy is diverted within the energy guide 122A to one or more of a plasma generator 233 (illustrated in FIG. 2) and the photoacoustic transducer 254 that is in optical communication with a side surface of the energy guide 122A. The photoacoustic transducer 254 then converts light energy into an acoustic wave that extends away from the side surface of the energy guide 122A.

The source manifold 136 can be positioned at or near the proximal portion 114 of the catheter system 100. The source manifold 136 can include one or more proximal end openings that can receive the one or more energy guides 122A of the energy guide bundle 122, the guidewire 112, and/or an inflation conduit 140 that is coupled in fluid communication with the fluid pump 138. The catheter system 100 can also include the fluid pump 138 that is configured to provide the catheter fluid 132 to the treatment assembly 104, i.e. via the inflation conduit 140, as needed. The fluid pump 138 can further be connected to the handle assembly 128 so that the handle assembly 128 can be used to control the fluid flow from the fluid pump through the catheter 102 and the assembly shaft 104A and into the director interior 146 of the energy director 104B.

As noted above, in the embodiment illustrated in FIG. 1, the system console 123 includes one or more of the energy source 124, the power source 125, the system controller 126, and the GUI 127. Alternatively, the system console 123 can include more components or fewer components than those specifically illustrated in FIG. 1. For example, in certain non-exclusive alternative embodiments, the system console 123 can be designed without the GUI 127. Still alternatively, one or more of the energy source 124, the power source 125, the system controller 126, and the GUI 127 can be provided within the catheter system 100 without the specific need for the system console 123.

As shown, the system console 123, and the components included therewith, is operatively coupled to the catheter 102, the energy guide bundle 122, and the remainder of the catheter system 100. For example, in some embodiments, as illustrated in FIG. 1, the system console 123 can include a console connection aperture 148 (also sometimes referred to generally as a “socket”) by which the energy guide bundle 122 is mechanically coupled to the system console 123. In such embodiments, the energy guide bundle 122 can include a guide coupling housing 150 (also sometimes referred to generally as a “ferrule”) that houses a portion, e.g., the guide proximal end 122P, of each of the energy guides 122A. The guide coupling housing 150 is configured to fit and be selectively retained within the console connection aperture 148 to provide the mechanical coupling between the energy guide bundle 122 and the system console 123.

The energy guide bundle 122 can also include a guide bundler 152 (or “shell”) that brings each of the individual energy guides 122A closer together so that the energy guides 122A and/or the energy guide bundle 122 can be in a more compact form as it extends with the catheter 102 adjacent to the heart valve 108 during use of the catheter system 100.

The energy source 124 can be selectively and/or alternatively coupled in optical communication with each of the energy guides 122A, i.e. to the guide proximal end 122P of each of the energy guides 122A, in the energy guide bundle 122. In particular, the energy source 124 is configured to generate energy in the form of a source beam 124A, such as a pulsed source beam, that can be selectively and/or alternatively directed to and received by each of the energy guides 122A in the energy guide bundle 122 as an individual guide beam 124B. Alternatively, the catheter system 100 can include more than one energy source 124. For example, in one non-exclusive alternative embodiment, the catheter system 100 can include a separate energy source 124 for each of the energy guides 122A in the energy guide bundle 122.

The energy source 124 can have any suitable design. In certain embodiments, the energy source 124 can be configured to provide sub-millisecond pulses of energy from the energy source 124 that are focused onto a small spot in order to couple it into the guide proximal end 122P of the energy guide 122A. Such pulses of energy are then directed and/or guided along the energy guides 122A to a location within the director interior 146 of the energy director 104B, thereby inducing plasma formation in the catheter fluid 132 within the director interior 146 of the energy director 104B, e.g., via the plasma generator 233 that can be located at or near the guide distal end 122D of the energy guide 122A. In particular, the energy emitted at the guide distal end 122D of the energy guide 122A energizes the plasma generator 233 to form the plasma within the catheter fluid 132 within the director interior 146. The plasma formation causes rapid bubble formation, and imparts pressure waves upon the treatment site 106. An exemplary plasma-induced bubble 134 is illustrated in FIG. 1.

As noted, the energy director 104B is configured to direct and/or guide energy in the form of the pressure waves, which are formed from the plasma and plasma bubble 134 generation within the director interior 146, toward the vascular lesions 106A at the treatment site 106 to enhance the delivery of such energy to the treatment site 106. By directing and/or guiding the energy in such manner, the energy director 104B imparts pressure onto and induces fractures in the vascular lesions 106A at the treatment site 106 within or adjacent to the heart valve 108. Thus, the energy director 104B and/or the treatment assembly 104 is able to effectively improve the efficacy of the catheter system 100.

In some embodiments, more than one energy guide 122A can be positioned within the director interior 146 of the energy director 104B. With such design, bubble 134 size and dynamics can be increased. Additionally, in certain embodiments, the energy guide(s) 122A can be steerable such that the guide distal end 122D of the energy guide 122A can be positioned at different locations within the director interior 146 so that the energy director 104B can more accurately direct the energy in the form of pressure waves toward the vascular lesions 106A at the treatment site 106.

In various non-exclusive alternative embodiments, the sub-millisecond pulses of energy from the energy source 124 can be delivered to the treatment site 106 at a frequency of between approximately one hertz (Hz) and 5000 Hz, between approximately 30 Hz and 1000 Hz, between approximately ten Hz and 100 Hz, or between approximately one Hz and 30 Hz. Alternatively, the sub-millisecond pulses of energy can be delivered to the treatment site 106 at a frequency that can be greater than 5000 Hz or less than one Hz, or any other suitable range of frequencies.

It is appreciated that although the energy source 124 is typically utilized to provide pulses of energy, the energy source 124 can still be described as providing a single source beam 124A, i.e. a single pulsed source beam.

The energy sources 124 suitable for use can include various types of light sources including lasers and lamps. Alternatively, the energy sources 124 can include any suitable type of energy source.

Suitable lasers can include short pulse lasers on the sub-millisecond timescale. In some embodiments, the energy source 124 can include lasers on the nanosecond (ns) timescale. The lasers can also include short pulse lasers on the picosecond (ps), femtosecond (fs), and microsecond (us) timescales. It is appreciated that there are many combinations of laser wavelengths, pulse widths and energy levels that can be employed to achieve plasma in the catheter fluid 132 of the catheter 102. In various non-exclusive alternative embodiments, the pulse widths can include those falling within a range including from at least ten ns to 3000 ns, at least 20 ns to 100 ns, or at least one ns to 500 ns. Alternatively, any other suitable pulse width range can be used.

Exemplary nanosecond lasers can include those within the UV to IR spectrum, spanning wavelengths of about ten nanometers (nm) to one millimeter (mm). In some embodiments, the energy sources 124 suitable for use in the catheter systems 100 can include those capable of producing light at wavelengths of from at least 750 nm to 2000 nm. In other embodiments, the energy sources 124 can include those capable of producing light at wavelengths of from at least 700 nm to 3000 nm. In still other embodiments, the energy sources 124 can include those capable of producing light at wavelengths of from at least 100 nm to ten micrometers (μm). Nanosecond lasers can include those having repetition rates of up to 200 kHz.

In some embodiments, the laser can include a Q-switched thulium:yttrium-aluminum-garnet (Tm:YAG) laser. In other embodiments, the laser can include a neodymium:yttrium-aluminum-garnet (Nd:YAG) laser, holmium:yttrium-aluminum-garnet (Ho:YAG) laser, erbium:yttrium-aluminum-garnet (Er:YAG) laser, excimer laser, helium-neon laser, carbon dioxide laser, as well as doped, pulsed, fiber lasers.

The catheter system 100 can generate pressure waves having maximum pressures in the range of at least one megapascal (MPa) to 100 MPa. The maximum pressure generated by a particular catheter system 100 will depend on the energy source 124, the absorbing material, the bubble expansion, the propagation medium, the energy director material, and other factors. In various non-exclusive alternative embodiments, the catheter systems 100 can generate pressure waves having maximum pressures in the range of at least approximately two MPa to 50 MPa, at least approximately two MPa to 30 MPa, or approximately at least 15 MPa to 25 MPa.

The pressure waves can be imparted upon the treatment site 106 from a distance within a range from at least approximately 0.1 millimeters (mm) to greater than approximately 25 mm extending from the energy guides 122A when the catheter 102 is placed at the treatment site 106. In various non-exclusive alternative embodiments, the pressure waves can be imparted upon the treatment site 106 from a distance within a range from at least approximately ten mm to 20 mm, at least approximately one mm to ten mm, at least approximately 1.5 mm to four mm, or at least approximately 0.1 mm to ten mm extending from the energy guides 122A when the catheter 102 is placed at the treatment site 106. In other embodiments, the pressure waves can be imparted upon the treatment site 106 from another suitable distance that is different than the foregoing ranges. In some embodiments, the pressure waves can be imparted upon the treatment site 106 within a range of at least approximately two MPa to 30 MPa at a distance from at least approximately 0.1 mm to ten mm. In some embodiments, the pressure waves can be imparted upon the treatment site 106 from a range of at least approximately two MPa to 25 MPa at a distance from at least approximately 0.1 mm to ten mm. Still alternatively, other suitable pressure ranges and distances can be used.

The power source 125 is electrically coupled to and is configured to provide necessary power to each of the energy source 124, the system controller 126, the GUI 127, and the handle assembly 128. The power source 125 can have any suitable design for such purposes.

The system controller 126 is electrically coupled to and receives power from the power source 125. Additionally, the system controller 126 is coupled to and is configured to control operation of each of the energy source 124 and the GUI 127. The system controller 126 can include one or more processors or circuits for purposes of controlling the operation of at least the energy source 124 and the GUI 127. For example, the system controller 126 can control the energy source 124 for generating pulses of energy as desired and/or at any desired firing rate. Additionally, the system controller 126 can operate to effectively and efficiently provide the desired fracture forces adjacent to and/or on or between adjacent leaflets 1086 within the heart valve 108 at the treatment site 106.

The system controller 126 can also be configured to control operation of other components of the catheter system 100 such as the positioning of the catheter 102 and/or the treatment assembly 104 adjacent to the treatment site 106, the deployment and expansion of the energy director 104B, etc. Further, or in the alternative, the catheter system 100 can include one or more additional controllers that can be positioned in any suitable manner for purposes of controlling the various operations of the catheter system 100. For example, in certain embodiments, an additional controller and/or a portion of the system controller 126 can be positioned and/or incorporated within the handle assembly 128.

The GUI 127 is accessible by the user or operator of the catheter system 100. Additionally, the GUI 127 is electrically connected to the system controller 126. With such design, the GUI 127 can be used by the user or operator to ensure that the catheter system 100 is effectively utilized to impart pressure onto and induce fractures into the vascular lesions 106A at the treatment site 106. The GUI 127 can provide the user or operator with information that can be used before, during and after use of the catheter system 100. In one embodiment, the GUI 127 can provide static visual data and/or information to the user or operator. In addition, or in the alternative, the GUI 127 can provide dynamic visual data and/or information to the user or operator, such as video data or any other data that changes over time during use of the catheter system 100. In various embodiments, the GUI 127 can include one or more colors, different sizes, varying brightness, etc., that may act as alerts to the user or operator. Additionally, or in the alternative, the GUI 127 can provide audio data or information to the user or operator. The specifics of the GUI 127 can vary depending upon the design requirements of the catheter system 100, or the specific needs, specifications and/or desires of the user or operator.

As shown in FIG. 1, the handle assembly 128 can be positioned at or near the proximal portion 114 of the catheter system 100, and/or near the source manifold 136. In this embodiment, the handle assembly 128 is coupled to the treatment assembly 104 and is positioned spaced apart from the energy director 104B. Alternatively, the handle assembly 128 can be positioned at another suitable location.

The handle assembly 128 is handled and used by the user or operator to operate, position and control the catheter 102 and/or the treatment assembly 104. The design and specific features of the handle assembly 128 can vary to suit the design requirements of the catheter system 100. In the embodiment illustrated in FIG. 1, the handle assembly 128 is separate from, but in electrical and/or fluid communication with one or more of the system controller 126, the energy source 124, the fluid pump 138, and the GUI 127. In some embodiments, the handle assembly 128 can integrate and/or include at least a portion of the system controller 126 within an interior of the handle assembly 128. For example, as shown, in certain such embodiments, the handle assembly 128 can include circuitry 156 that can form at least a portion of the system controller 126. In one embodiment, the circuitry 156 can include a printed circuit board having one or more integrated circuits, or any other suitable circuitry. In an alternative embodiment, the circuitry 156 can be omitted, or can be included within the system controller 126, which in various embodiments can be positioned outside of the handle assembly 128, e.g., within the system console 123. It is understood that the handle assembly 128 can include fewer or additional components than those specifically illustrated and described herein.

FIG. 2 is a simplified schematic view of a portion of the heart valve 108, including two leaflets 108B, and a portion of an embodiment of the valvular lithotripsy treatment assembly 204 that can be used within the catheter system 100 (illustrated in FIG. 1). As above, the treatment assembly 204 is usable for treating one or more vascular lesions 106A at the treatment site 106 within and/or adjacent to the heart valve 108. For example, as shown in FIG. 2, the treatment assembly 204 can be used to treat one or more vascular lesions 106A that are formed onto and/or adjacent to the leaflets 108B of the heart valve 108 at the treatment site 106.

The design of the treatment assembly 204 can be varied to suit the requirements of the user of the catheter system 100. As illustrated, in various embodiments, the treatment assembly 204 includes an assembly shaft 204A, and an energy director 204B that is coupled to and/or secured to the assembly shaft 204A. In some embodiments, the treatment assembly 204 and/or the energy director 204B are movable between a retracted position (and collapsed state) and a deployed position (and expanded state). The deployed position and expanded state for the treatment assembly 204 and/or the energy director 204B is illustrated in FIG. 2. As shown, when the treatment assembly 204 and/or the energy director 204B are in the deployed position and the expanded state, a director distal end 204D of the energy director 204B can be positioned substantially adjacent to the vascular lesions 106A at the treatment site 106.

The assembly shaft 204A can have any suitable shape. For example, in some embodiments, the assembly shaft 204A can be substantially cylindrical tube-shaped. Alternatively, the assembly shaft 204A can be substantially rectangular tube-shaped, square tube-shape, oval tube-shaped, or another suitable shape.

As shown, the assembly shaft 204A can define an inflation and/or irrigation lumen through which the catheter fluid 132 (illustrated in FIG. 1) can be transmitted to the energy director 204B. For example, as shown in FIG. 2, the assembly shaft 204A can include an inflation port 260 (illustrated in phantom) through which the catheter fluid 132 can be directed into a director interior 246 as defined by a director wall 230 of the energy director 204B. Additionally, the assembly shaft 104A can further define one or more energy guide lumens 262 (illustrated in phantom) through which the one or more energy guides 222A can extend.

As noted, the energy director 204B can include the director wall 230 that defines the director interior 246. During use of the treatment assembly 204, the energy director 204B is configured to receive and retain the catheter fluid 132 substantially within the director interior 246 of the energy director 204B. As above, in various embodiments, the retaining of the catheter fluid 132 within the director interior 246 of the energy director 204B enables the creation of a plasma within the director interior 246, and the energy director 204B is configured to direct the energy from the plasma, such as in the form of one or more plasma bubbles 234 and/or corresponding pressure waves, toward the vascular lesions 106A at the treatment site 106. Additionally, the catheter fluid 132 being directed into and retained within the director interior 246 of the energy director 204B is again also utilized to inhibit blood from entering into the director interior 246 so as to reduce the risk of blood coagulation.

The energy director 204B can be any suitable shape when the treatment assembly 204 and/or the energy director 204B is in the deployed position and the expanded state. More particularly, as shown, the energy director 204B can be substantially cone-shaped and can include a narrow, circular-shaped, director proximal end 204P that is coupled to the assembly shaft 204A, and a wider, circular-shaped, director distal end 204D that is positioned away from the assembly shaft 204A. In one embodiment, the director distal end 204D can be fully open. With such design, the energy director 204B is able to direct and/or guide energy through the open director distal end 204D toward the vascular lesions 106A at the treatment site 106. Alternatively, in other embodiments, the director distal end 204D can be partially closed with an aperture formed thereon. Still alternatively, the energy director 204B can be substantially spherical-shaped, or can have another suitable shape when in the deployed position and the expanded state.

As shown in this embodiment, the guide distal end 222D of one energy guide 222A is positioned within the director interior 246 of the energy director 204B as defined by the director wall 230. As such, the energy guide 222A is configured to guide energy from the energy source 124 (illustrated in FIG. 1) along the energy guide 222A and to the guide distal end 222D within the director interior 246.

Additionally, as shown, the energy guide 222A can include and/or incorporate a photoacoustic transducer 254 and/or a plasma generator 233 at or near the guide distal end 222D that is configured to utilize the energy that is guided through the energy guide 222A to induce plasma formation in the catheter fluid 132 within the director interior 246 of the energy director 204B. In particular, the energy emitted at the guide distal end 222D of the energy guide 222A energizes the plasma generator 233 and/or the photoacoustic transducer 254 to form the plasma within the catheter fluid 132 within the director interior 246. The plasma formation causes rapid bubble 234 formation, and thus imparts pressure waves upon the treatment site 106.

Thus, in this embodiment, with the cone-shaped design, the energy director 204B is configured to direct and/or guide energy in the form of the pressure waves, which are formed from the plasma and plasma bubble 234 generation within the director interior 246, toward the vascular lesions 106A at the treatment site 106 to enhance the delivery of such energy to the treatment site 106. By directing and/or guiding the energy in such manner, the energy director 204B imparts pressure onto and induces fractures in the vascular lesions 106A at the treatment site 106 within or adjacent to the heart valve 108. Thus, the energy director 204B and/or the treatment assembly 204 is able to effectively improve the efficacy of the catheter system 100.

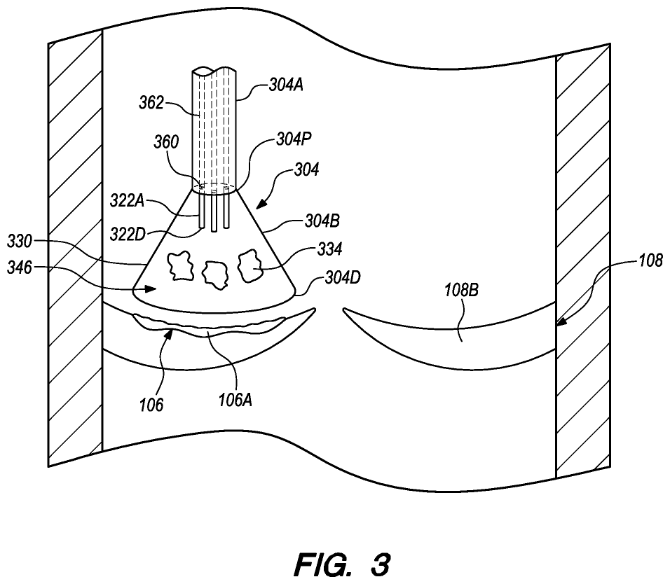

FIG. 3 is a simplified schematic view of a portion of the heart valve 108, including two leaflets 108B, and a portion of another embodiment of the valvular lithotripsy treatment assembly 304 that can be used within the catheter system 100 (illustrated in FIG. 1). As above, the treatment assembly 304 is again usable for treating one or more vascular lesions 106A at the treatment site 106 within and/or adjacent to the heart valve 108. For example, as shown in FIG. 3, the treatment assembly 304 can be used to treat one or more vascular lesions 106A that are formed onto and/or adjacent to the leaflets 1086 of the heart valve 108 at the treatment site 106.

In this embodiment, the treatment assembly 304 is somewhat similar to the previous embodiments illustrated and described herein above. For example, in the embodiment illustrated in FIG. 3, the treatment assembly 304 again includes an assembly shaft 304A, and an energy director 304B that is coupled to and/or secured to the assembly shaft 304A. Additionally, in some embodiments, the treatment assembly 304 and/or the energy director 304B are movable between a retracted position (and collapsed state) and a deployed position (and expanded state). The deployed position and expanded state for the treatment assembly 304 and/or the energy director 304B is illustrated in FIG. 3. As shown, when the treatment assembly 304 and/or the energy director 304B are in the deployed position and the expanded state, a director distal end 304D of the energy director 304B can be positioned substantially adjacent to the vascular lesions 106A at the treatment site 106.

The assembly shaft 304A and the energy director 304B are substantially similar to the embodiments illustrated and described herein above. For example, the assembly shaft 304A can again include one or more inflation ports 360 (illustrated in phantom) through which the catheter fluid 132 (illustrated in FIG. 1) can be directed into a director interior 346 as defined by a director wall 330 of the energy director 304B; and the assembly shaft 304A can again define one or more energy guide lumens 362 (illustrated in phantom) through which the one or more energy guides 322A can extend. During use of the treatment assembly 304, the energy director 304B is again configured to receive and retain the catheter fluid 132 substantially within the director interior 346 of the energy director 304B, which enables the creation of a plasma within the director interior 346. The energy director 304B is further configured to direct the energy from the plasma, such as in the form of one or more plasma bubbles 334 and/or corresponding pressure waves, toward the vascular lesions 106A at the treatment site 106. Additionally, in this embodiment, the energy director 304B can again be substantially cone-shaped and can include a narrow, circular-shaped, director proximal end 304P that is coupled to the assembly shaft 304A, and a wider, circular-shaped, director distal end 304D that is positioned away from the assembly shaft 304A. Further, in one embodiment, the director distal end 304D can again be fully open toward the vascular lesions 106A when the energy director 304 is positioned with the director distal end 304D positioned substantially adjacent to the vascular lesions 106A at the treatment site 106.

However, in the embodiment illustrated in FIG. 3, a plurality of energy guides 322A, three in this particular example, can be used in conjunction with the treatment assembly 304. In particular, in this embodiment, the guide distal ends 322D of three energy guides 322A are positioned within the director interior 346 of the energy director 304B as defined by the director wall 330. As such, the energy guides 322A are configured to guide energy from the energy source 124 (illustrated in FIG. 1) along the energy guides 322A and to the guide distal ends 322D within the director interior 346. With such design, the size and dynamics of the plasma bubbles 334 that are created within the director interior 346 of the energy director 304B increased to more effectively and efficiently impart pressure onto and induces fractures in the vascular lesions 106A at the treatment site 106 within or adjacent to the heart valve 108. It is appreciated that the guide distal ends 322A of any suitable number of energy guides 322A can be positioned within the director interior 346 of the energy director 304B for purposes of imparting pressure onto and inducing fractures in the vascular lesions 106A at the treatment site 106 within or adjacent to the heart valve 108.

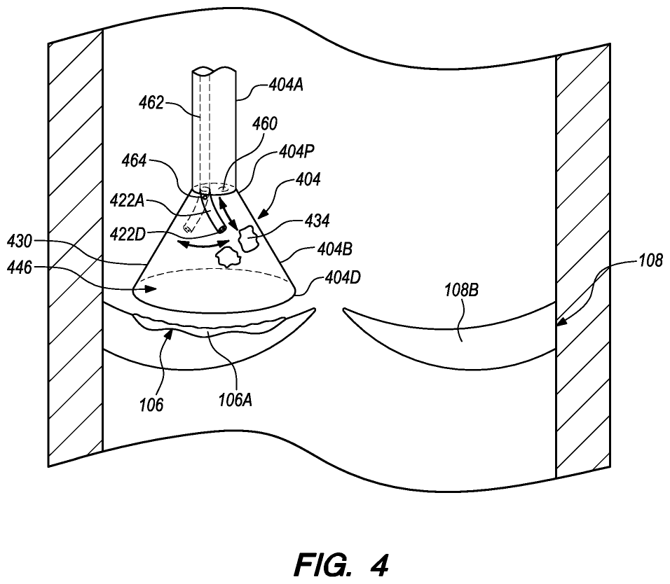

FIG. 4 is a simplified schematic view of a portion of the heart valve 108, including two leaflets 108B, and a portion of still another embodiment of the valvular lithotripsy treatment assembly 404 that can be used within the catheter system 100 (illustrated in FIG. 1). As above, the treatment assembly 404 is again usable for treating one or more vascular lesions 106A at a treatment site 106 within and/or adjacent to the heart valve 108. For example, as shown in FIG. 4, the treatment assembly 404 can be used to treat one or more vascular lesions 106A that are formed onto and/or adjacent to the leaflets 1086 of the heart valve 108 at the treatment site 106.

As illustrated, the treatment assembly 404 is again somewhat similar to the previous embodiments illustrated and described herein above. For example, in the embodiment illustrated in FIG. 4, the treatment assembly 404 again includes an assembly shaft 404A, and an energy director 404B that is coupled to and/or secured to the assembly shaft 404A. Additionally, in some embodiments, the treatment assembly 404 and/or the energy director 404B are movable between a retracted position (and collapsed state) and a deployed position (and expanded state). The deployed position and expanded state for the treatment assembly 404 and/or the energy director 404B is illustrated in FIG. 4. As shown, when the treatment assembly 404 and/or the energy director 404B are in the deployed position and the expanded state, a director distal end 404D of the energy director 404B can be positioned substantially adjacent to the vascular lesions 106A at the treatment site 106.

The assembly shaft 404A and the energy director 404B are substantially similar to the embodiments illustrated and described herein above. For example, the assembly shaft 404A can again include one or more inflation ports 460 through which the catheter fluid 132 (illustrated in FIG. 1) can be directed into a director interior 446 as defined by a director wall 430 of the energy director 404B; and the assembly shaft 404A can again define one or more energy guide lumens 462 through which the one or more energy guides 422A can extend. During use of the treatment assembly 404, the energy director 404B is again configured to receive and retain the catheter fluid 132 substantially within the director interior 446 of the energy director 404B, which enables the creation of a plasma within the director interior 446. The energy director 404B is further configured to direct the energy from the plasma, such as in the form of one or more plasma bubbles 434 and/or corresponding pressure waves, toward the vascular lesions 106A at the treatment site 106. Additionally, in this embodiment, the energy director 404B can again be substantially cone-shaped and can include a narrow, circular-shaped, director proximal end 404P that is coupled to the assembly shaft 404A, and a wider, circular-shaped, director distal end 404D that is positioned away from the assembly shaft 404A. Further, in one embodiment, the director distal end 404D can again be fully open toward the vascular lesions 106A when the energy director 404 is positioned with the director distal end 404D positioned substantially adjacent to the vascular lesions 106A at the treatment site 106.

However, in this embodiment, the guide distal end 422D of the energy guide 422A is steerable within the director interior 446 so that the plasma generated within the director interior 446 can be better and more accurately directed toward the vascular lesions 106A at the treatment site 106 within or adjacent to the heart valve 108. More specifically, in certain such embodiments, a steering member 464 can be coupled to the energy guide 422A so that the guide distal end 422D can be steered and positioned as desired. In one such embodiment, the steering member 464 can be configured to steer the guide distal end 422D of the energy guide 422A such that the guide distal end 422D is able to trace a substantially circular path within the director interior 446. Additionally, in some embodiments, the guide distal end 422D can be angled and/or bent relative to a length of the energy guide to better enable the guide distal end 422D to trace such a substantially circular path. Alternatively, the steering member 464 can be configured to steer the guide distal end 422D of the energy guide 422A in another suitable manner.

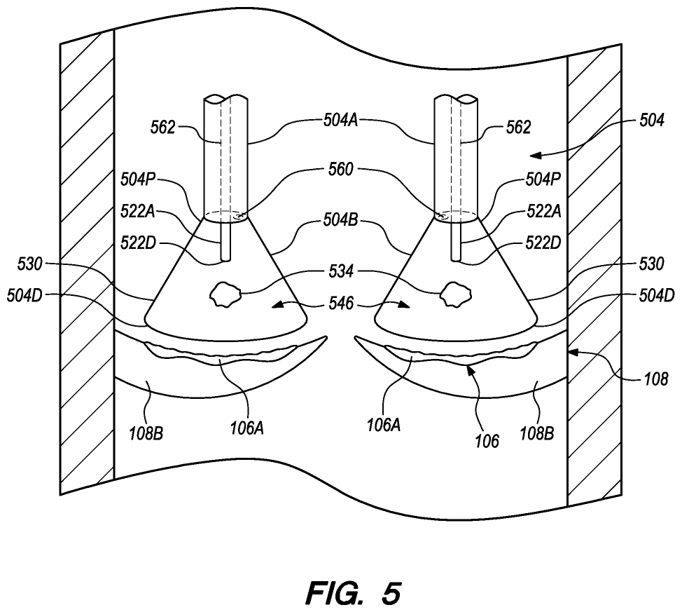

FIG. 5 is a simplified schematic view of a portion of the heart valve 108, including two leaflets 108B, and a portion of another embodiment of the valvular lithotripsy treatment assembly 504 that can be used within the catheter system 100 (illustrated in FIG. 1). As above, the treatment assembly 504 is again usable for treating one or more vascular lesions 106A at the treatment site 106 within and/or adjacent to the heart valve 108. For example, as shown in FIG. 5, the treatment assembly 504 can be used to treat one or more vascular lesions 106A that are formed onto and/or adjacent to the leaflets 1086 of the heart valve 108 at the treatment site 106.

As illustrated, the treatment assembly 504 is again somewhat similar to the previous embodiments illustrated and described herein above. For example, in the embodiment illustrated in FIG. 5, the treatment assembly 504 again includes an assembly shaft 504A, and an energy director 504B that is coupled to and/or secured to the assembly shaft 504A that are substantially similar to what has been illustrated and described in relation to previous embodiments. However, in this embodiment, the treatment assembly 504 further includes a second assembly shaft 504A, and a second energy director 504B that is coupled to and/or secured to the second assembly shaft 504A. Stated in another manner, in this embodiment, the catheter system 100 can be said to include a first treatment assembly 504 and a second treatment assembly 504 that each include an assembly shaft 504A and an energy director 504B that is coupled to and/or secured to the assembly shaft 504A. In one embodiment, each of the treatment assemblies 504 can be substantially identical to one another. Alternatively, in another embodiment, the treatment assemblies 504 can be configured to have a different design from one another. Still alternatively, in still another embodiment, it is appreciated that the catheter system 100 can include more than two, e.g., three, such treatment assemblies 504.

In some embodiments, each of the treatment assemblies 504 and/or energy directors 504B are movable between a retracted position (and collapsed state) and a deployed position (and expanded state). The deployed position and expanded state for the treatment assemblies 504 and/or the energy directors 504B is illustrated in FIG. 5. As shown, when the treatment assemblies 504 and/or the energy directors 504B are in the deployed position and the expanded state, a director distal end 504D of each energy director 504B can be positioned substantially adjacent to the vascular lesions 106A at the treatment site 106.

In the embodiment illustrated in FIG. 5, the assembly shaft 504A and the energy director 504B of each of the treatment assemblies 504 can be substantially similar to the embodiments illustrated and described herein above. For example, for each treatment assembly 504, the assembly shaft 304A can again include one or more inflation ports 560 (illustrated in phantom) through which the catheter fluid 132 (illustrated in FIG. 1) can be directed into a director interior 546 as defined by a director wall 530 of the energy director 504B; and the assembly shaft 504A can again define one or more energy guide lumens 562 (illustrated in phantom) through which the one or more energy guides 522A can extend. During use of each treatment assembly 504, the energy director 504B is again configured to receive and retain the catheter fluid 132 substantially within the director interior 546 of the energy director 504B, which enables the creation of a plasma within the director interior 546. The energy director 504B is further configured to direct the energy from the plasma, such as in the form of one or more plasma bubbles 534 and/or corresponding pressure waves generated near the guide distal end 522D of the energy guide 522A, toward the vascular lesions 106A at the treatment site 106. Additionally, in this embodiment, the energy director 504B can again be substantially cone-shaped and can include a narrow, circular-shaped, director proximal end 504P that is coupled to the assembly shaft 504A, and a wider, circular-shaped, director distal end 504D that is positioned away from the assembly shaft 504A. Further, in one embodiment, the director distal end 504D can again be fully open toward the vascular lesions 106A when the energy director 504 is positioned with the director distal end 504D positioned substantially adjacent to the vascular lesions 106A at the treatment site 106.

With the design illustrated in FIG. 5, each of the individual treatment assemblies 504, and/or each of the assembly shafts 504A and corresponding energy directors 504B, can be configured to direct energy from the plasma generated within the director interior 546, such as in the form of one or more plasma bubbles 534 and/or corresponding pressure waves, toward vascular lesions 106A formed on and/or adjacent to separate leaflets 1086 of the heart valve 108.

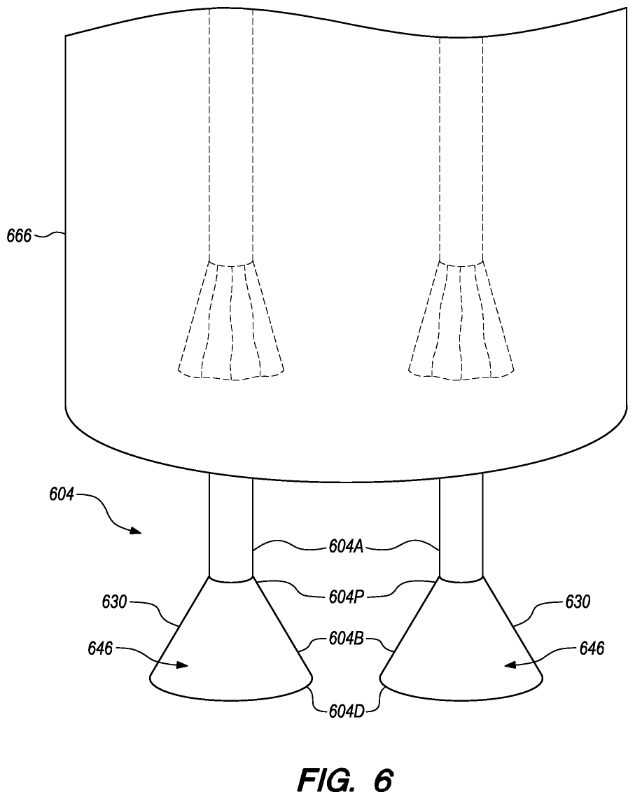

FIG. 6 is a simplified schematic view of a portion of yet another embodiment of the valvular lithotripsy treatment assembly 604 that can be used within the catheter system 100 (illustrated in FIG. 1). As above, the treatment assembly 604 is again usable for treating one or more vascular lesions 106A (illustrated in FIG. 1) at the treatment site 106 (illustrated in FIG. 1) within and/or adjacent to a heart valve 108 (illustrated in FIG. 1).

As illustrated, the treatment assembly 604 is somewhat similar to the embodiment illustrated and described in relation to FIG. 5. In particular, as illustrated in this embodiment, the treatment assembly 604 again includes a first assembly shaft 604A, a first energy director 604B that is coupled to and/or secured to the first assembly shaft 604A, a second assembly shaft 604A, and a second energy director 604B that is coupled to and/or secured to the second assembly shaft 604A. Stated in another manner, the catheter system 100 can again include two treatment assemblies 604, with each treatment assembly 604 including an assembly shaft 604A, and an energy director 604B that is coupled to and/or secured to the assembly shaft 604A. As with the previous embodiment, each of the individual treatment assemblies 604, and/or each of the assembly shafts 604A and corresponding energy directors 604B, can be configured to direct energy from the plasma generated in the catheter fluid 132 (illustrated in FIG. 1) within the director interior 646 as defined by the director wall 630 of each of the energy directors 604B toward the vascular lesions 106A formed on and/or adjacent to separate leaflets 108B (illustrated in FIG. 1) of the heart valve 108. Additionally, in this embodiment, the energy director 604B can again be substantially cone-shaped and can include a narrow, circular-shaped, director proximal end 604P that is coupled to the assembly shaft 604A, and a wider, circular-shaped, director distal end 604D that is positioned away from the assembly shaft 604A.

In this embodiment, the treatment assemblies 604 are further illustrated as being retained within a single outer sheath 666. Alternatively, in another embodiment, each of the individual treatment assemblies 604 can be retained within separate sheaths.

As with the previous embodiments, each of the treatment assemblies 604 and/or energy directors 604B are movable between a retracted position (and collapsed state) and a deployed position (and expanded state). The retracted position and collapsed state for the treatment assemblies 604 and/or the energy directors 604 is illustrated in dashed lines in FIG. 6; and the deployed position and expanded state for the treatment assemblies 604 and/or the energy directors 604B is illustrated in solid lines in FIG. 6. As with previous embodiments, when the treatment assemblies 604 and/or the energy directors 604B are in the deployed position and the expanded state, the director distal end 604D of each energy director 604B can be positioned substantially adjacent to the vascular lesions 106A at the treatment site 106.

As illustrated in FIG. 6, when the treatment assemblies 604 and/or the energy directors 604B are in the retracted position and the collapsed state, the treatment assemblies 604 and/or the energy directors 604B are positioned at least substantially, if not entirely, within the outer sheath 666. Conversely, when the treatment assemblies 604 and/or the energy directors 604B are in the deployed position and the expanded state, the treatment assemblies 604 and/or the energy directors 604B are positioned to extend outside of and/or away from the outer sheath 666.

It is appreciated that certain components of the catheter system 100 that are shown in other embodiments and that are used as part of and/or in conjunction with the treatment assembly 604, such as the energy guides 122A (illustrated in FIG. 1), the inflation ports 260 (illustrated in FIG. 2) and the energy guide lumens 262 (illustrated in FIG. 2), are not illustrated in FIG. 6 for purposes of clarity. However, such components would likely be included in any implementation of this embodiment of the treatment assembly 604.

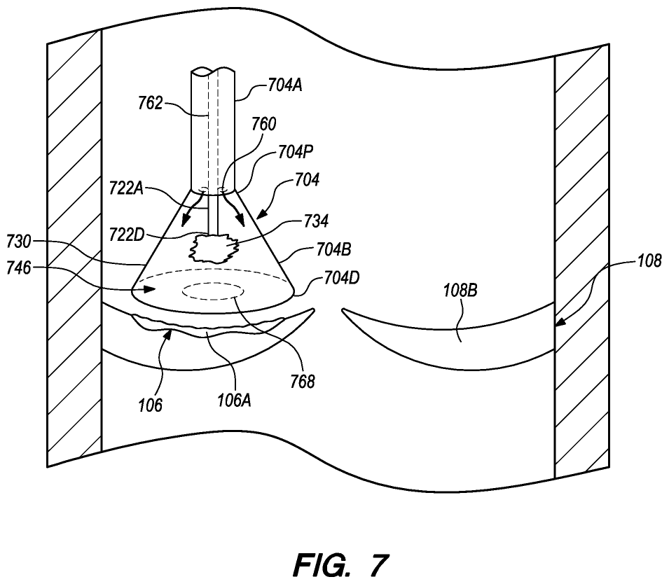

FIG. 7 is a simplified schematic view of a portion of the heart valve 108, including two leaflets 108A, and a portion of another embodiment of the valvular lithotripsy treatment assembly 704 that can be used within the catheter system 100 (illustrated in FIG. 1). As above, the treatment assembly 704 is again usable for treating one or more vascular lesions 106A at the treatment site 106 within and/or adjacent to the heart valve 108. For example, as shown in FIG. 7, the treatment assembly 704 can be used to treat one or more vascular lesions 106A that are formed onto and/or adjacent to the leaflets 1086 of the heart valve 108 at the treatment site 106.

As illustrated, the treatment assembly 704 is again somewhat similar to the previous embodiments illustrated and described herein above. For example, in the embodiment illustrated in FIG. 7, the treatment assembly 704 again includes an assembly shaft 704A, and an energy director 704B that is coupled to and/or secured to the assembly shaft 704A. Additionally, in some embodiments, the treatment assembly 704 and/or the energy director 704B are movable between a retracted position (and collapsed state) and a deployed position (and expanded state). The deployed position and expanded state for the treatment assembly 704 and/or the energy director 704B is illustrated in FIG. 7. As shown, when the treatment assembly 704 and/or the energy director 704B are in the deployed position and the expanded state, a director distal end 704D of the energy director 704B can be positioned substantially adjacent to the vascular lesions 106A at the treatment site 106.

The assembly shaft 704A and the energy director 704B are substantially similar to the embodiments illustrated and described herein above. For example, the assembly shaft 704A can again include one or more inflation ports 760 through which the catheter fluid 132 (illustrated in FIG. 1) can be directed into a director interior 746 as defined by a director wall 730 of the energy director 704B; and the assembly shaft 704A can again define one or more energy guide lumens 762 through which the one or more energy guides 722A can extend. Additionally, as shown, the guide distal end 722D of the energy guide 722A is positioned within the director interior 746 of the energy director 704B as defined by the director wall 730. As such, the energy guide 722A is again configured to guide energy from the energy source 124 (illustrated in FIG. 1) along the energy guide 722A and to the guide distal end 722D within the director interior 746.

During use of the treatment assembly 704, the energy director 704B is again configured to receive and retain the catheter fluid 132 substantially within the director interior 746 of the energy director 704B. With the energy guided by the energy guide 722A into the director interior 746, a plasma is created in the catheter fluid 132 within the director interior 746. The energy director 704B is further configured to direct the energy from the plasma, such as in the form of one or more plasma bubbles 734 and/or corresponding pressure waves, toward the vascular lesions 106A at the treatment site 106.

In this embodiment, the energy director 704B can again be substantially cone-shaped and can include a narrow, circular-shaped, director proximal end 704P that is coupled to the assembly shaft 704A, and a wider, circular-shaped, director distal end 704D that is positioned away from the assembly shaft 704A. However, in this embodiment, the energy director 704B further includes a director aperture 768 (illustrated in phantom) that is formed into the director distal end 704D of the energy director 704B. With such design, the energy from the plasma formed in the catheter fluid 132 within the director interior 746, such as in the form of the one or more plasma bubbles 734 and/or corresponding pressure waves, can be more precisely and accurately directed toward the vascular lesions 106A on and/or adjacent to the leaflets 1086 of the heart valve 108 at the treatment site 106. The director aperture 768 can further help maintain a desired fluid pressure for the catheter fluid 132 within the director interior 746, so as to further assist the energy director 704B in maintaining its desired expanded state. It is appreciated that the director aperture 768 can have any suitable size and shape for purposes of directing the plasma energy toward the vascular lesions 106A at the treatment site 106 as desired.

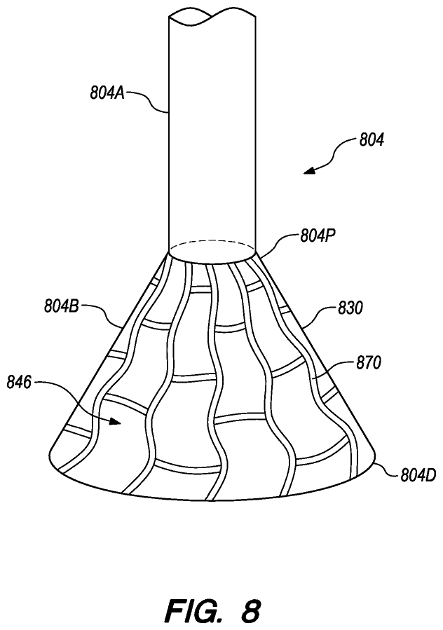

FIG. 8 is a simplified schematic view of a portion of still another embodiment of the valvular lithotripsy treatment assembly 804 that can be used within the catheter system 100 (illustrated in FIG. 1). As above, the treatment assembly 804 is again usable for treating one or more vascular lesions 106A (illustrated in FIG. 1) at a treatment site 106 (illustrated in FIG. 1) within and/or adjacent to a heart valve 108 (illustrated in FIG. 1).

As illustrated, the treatment assembly 804 is again somewhat similar to the previous embodiments illustrated and described herein above. For example, in the embodiment illustrated in FIG. 8, the treatment assembly 804 again includes an assembly shaft 804A, and an energy director 804B that is coupled to and/or secured to the assembly shaft 804A. As with the previous embodiments, the treatment assembly 804, and/or the assembly shaft 804A and corresponding energy director 804B, can be configured to direct energy from plasma generated in the catheter fluid 132 (illustrated in FIG. 1) within the director interior 846 as defined by the director wall 830 of the energy director 804B toward the vascular lesions 106A formed on and/or adjacent to separate leaflets 108B (illustrated in FIG. 1) of the heart valve 108. Additionally, in this embodiment, the energy director 804B can again be substantially cone-shaped and can include a narrow, circular-shaped, director proximal end 804P that is coupled to the assembly shaft 804A, and a wider, circular-shaped, director distal end 804D that is positioned away from the assembly shaft 804A. Further, in one embodiment, the director distal end 804D can again be fully open toward the vascular lesions 106A when the energy director 804 is positioned with the director distal end 804D positioned substantially adjacent to the vascular lesions 106A at the treatment site 106.

In some embodiments, the treatment assembly 804 and/or the energy director 804B are movable between a retracted position (and collapsed state) and a deployed position (and expanded state). The expanded state for the treatment assembly 804 and/or the energy director 804B is illustrated in FIG. 8.

In certain embodiments, as shown in FIG. 8, the treatment assembly 804 can further include an expansion assistance structure 870 that is coupled to the director wall 830 of the energy director 804B. In certain such embodiments, the expansion assistance structure 870 is self-expanding, such that when the treatment assembly 804 and/or the energy director 804B is moved to the deployed position, the expansion assistance structure 870 will automatically open up so that the energy director 804B and/or the director wall 830 can define its desired shape, i.e. substantially cone-shaped in this embodiment.

The design of the expansion assistance structure 870 can be varied as desired. In some embodiments, as shown, the expansion assistance structure 870 includes a lattice-like structure, with interwoven elements. Alternatively, the expansion assistance structure 870 can have another suitable design.

Additionally, the expansion assistance structure 870 can be formed from any suitable materials. For example, in certain non-exclusive alternative embodiments, the expansion assistance structure 870 can be formed from one or more of metallic materials, nitinol, plastic, or other suitable materials.

It is appreciated that certain components of the catheter system 100 that are shown in other embodiments and that are used as part of and/or in conjunction with the treatment assembly 804, such as the energy guides 122A (illustrated in FIG. 1), the inflation ports 260 (illustrated in FIG. 2) and the energy guide lumens 262 (illustrated in FIG. 2), are not illustrated in FIG. 8 for purposes of clarity. However, such components would likely be included in any implementation of this embodiment of the treatment assembly 804.

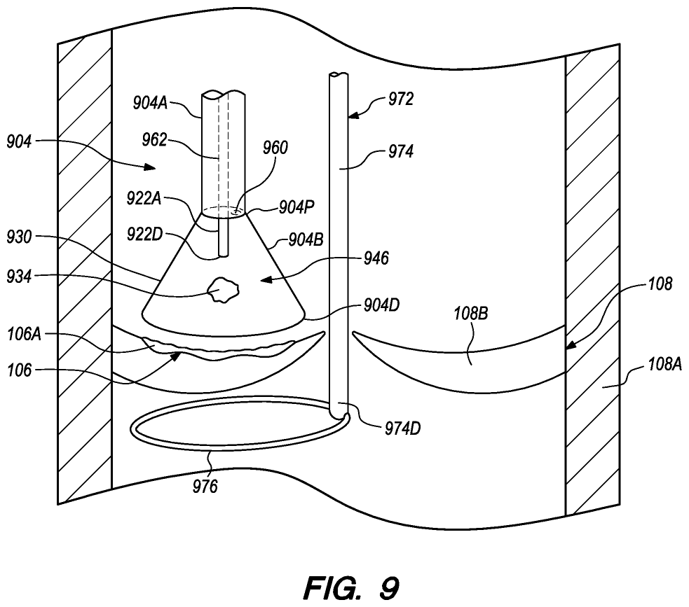

FIG. 9 is a simplified schematic view of a portion of the heart valve 108, including two leaflets 108B, and a portion of yet another embodiment of the valvular lithotripsy treatment assembly 904 that can be used within the catheter system 100 (illustrated in FIG. 1). As above, the treatment assembly 904 is again usable for treating one or more vascular lesions 106A at the treatment site 106 within and/or adjacent to the heart valve 108. For example, as shown in FIG. 9, the treatment assembly 904 can be used to treat one or more vascular lesions 106A that are formed onto and/or adjacent to the leaflets 1086 of the heart valve 108 at the treatment site 106.

As illustrated, the treatment assembly 904 is again somewhat similar to the previous embodiments illustrated and described herein above. For example, in the embodiment illustrated in FIG. 9, the treatment assembly 904 again includes an assembly shaft 904A, and an energy director 904B that is coupled to and/or secured to the assembly shaft 904A. Additionally, in some embodiments, the treatment assembly 904 and/or the energy director 904B are movable between a retracted position (and collapsed state) and a deployed position (and expanded state). The deployed position and expanded state for the treatment assembly 904 and/or the energy director 904B is illustrated in FIG. 9. As shown, when the treatment assembly 904 and/or the energy director 904B are in the deployed position and the expanded state, a director distal end 904D of the energy director 904B can be positioned substantially adjacent to the vascular lesions 106A at the treatment site 106.

The assembly shaft 904A and the energy director 904B are substantially similar to the embodiments illustrated and described herein above. For example, the assembly shaft 904A can again include one or more inflation ports 960 (illustrated in phantom) through which the catheter fluid 132 (illustrated in FIG. 1) can be directed into a director interior 946 as defined by a director wall 930 of the energy director 904B; and the assembly shaft 904A can again define one or more energy guide lumens 962 (illustrated in phantom) through which the one or more energy guides 922A can extend. During use of the treatment assembly 904, the energy director 904B is again configured to receive and retain the catheter fluid 132 substantially within the director interior 946 of the energy director 904B, which enables the creation of a plasma within the director interior 946. The energy director 904B is further configured to direct the energy from the plasma, such as in the form of one or more plasma bubbles 934 and/or corresponding pressure waves generated near the guide distal end 922D of the energy guide 922A, toward the vascular lesions 106A at the treatment site 106. Additionally, in this embodiment, the energy director 904B can again be substantially cone-shaped and can include a narrow, circular-shaped, director proximal end 904P that is coupled to the assembly shaft 904A, and a wider, circular-shaped, director distal end 904D that is positioned away from the assembly shaft 904A. Further, in one embodiment, the director distal end 904D can again be fully open toward the vascular lesions 106A when the energy director 904 is positioned with the director distal end 904D positioned substantially adjacent to the vascular lesions 106A at the treatment site 106.

However, in the embodiment illustrated in FIG. 9, the treatment assembly 904 further includes a leaflet support assembly 972 that is configured to support at least one of the leaflets 1086 relative to the energy director 904B. In particular, as illustrated, the leaflet support assembly 972 includes a support shaft 974, and a leaflet supporter 976 that is coupled to, secured to and/or integrally formed with the support shaft 974.

As shown, during use of the leaflet support assembly 972, the support shaft 974 is positioned to extend into the heart valve 108 and past the leaflets 108B. In certain embodiments, the support shaft 974 can be a thin, cylindrical shaft. Alternatively, the support shaft 974 can have another suitable size and/or shape.

As noted, the leaflet supporter 976 is coupled to, secured to and/or integrally formed with the support shaft 974. More particularly, as shown, the leaflet supporter 976 is configured to extend substantially transversely and/or perpendicularly away from a shaft distal end 974D of the support shaft 974. During treatment of the leaflets 108B, the support shaft 974 is extended through the heart valve 108 such that the shaft distal end 974D of the support shaft 974 is positioned on the opposite side of the leaflets 1086 from the energy director 904B. Thus, with the leaflet supporter 976 extending substantially transversely and/or perpendicularly away from the shaft distal end 974D of the support shaft 974, the leaflet supporter 976 is configured to be positioned adjacent to at least one of the leaflets 108A, on the opposite side of the leaflet 1086 as the energy director 904B. Accordingly, during treatment of the vascular lesions 106A at the treatment site 106 on and/or adjacent to the leaflets 1086 of the heart valve 108, the leaflet 1086 is effectively pinched between the leaflet supporter 976 and the energy director 904B. With such design, when the energy director 904B directs energy in the form of plasma bubbles 934 and/or corresponding pressure waves onto the leaflet 108B, the leaflet supporter 108B supports the leaflet 1086 and inhibits the leaflet 1086 from deflecting due to the pressure waves being against it. Thus, more of the force from the pressure waves is directly received by the vascular lesions 106A, thereby increasing the ability to induce fractures in the vascular lesions 106A at the treatment site 106.

The leaflet supporter 976 can have any suitable size and/or shape. For example, in certain non-exclusive embodiments, the leaflet supporter 976 can be somewhat flat, oval-shaped, and can extend from between the leaflets 108B to a point that is close to an outer edge of the leaflet 108B and near the valve wall 108A. Alternatively, the leaflet supporter 976 can have another suitable size and/or shape.

The leaflet support assembly 972 and/or the leaflet supporter 976 can be made from any suitable materials. For example, in some non-exclusive alternative embodiments, the leaflet support assembly 972 and/or the leaflet supporter 976 can be formed from one or more of metallic materials, nitinol, plastic, or other suitable materials.

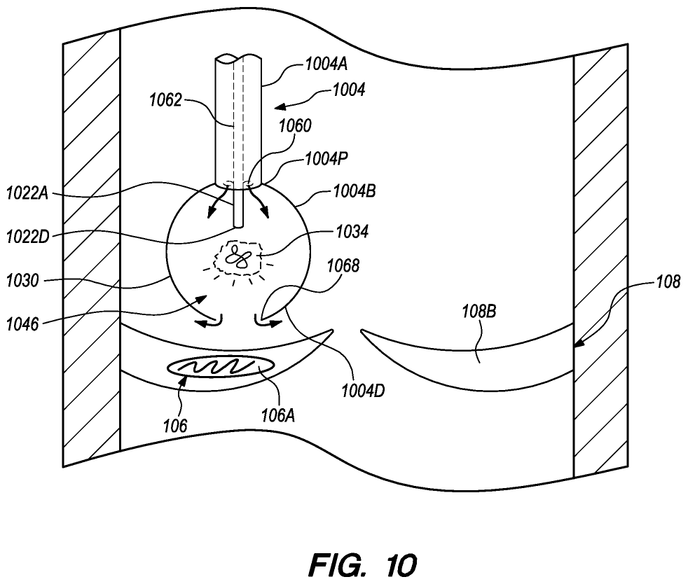

FIG. 10 is a simplified schematic view of a portion of the heart valve 108, including two leaflets 1086, and a portion of still yet another embodiment of the valvular lithotripsy treatment assembly 1004 that can be used within the catheter system 100 (illustrated in FIG. 1). As above, the treatment assembly 1004 is again usable for treating one or more vascular lesions 106A at the treatment site 106 within and/or adjacent to the heart valve 108. For example, as shown in FIG. 10, the treatment assembly 1004 can be used to treat one or more vascular lesions 106A that are formed onto and/or adjacent to the leaflets 1086 of the heart valve 108 at the treatment site 106.

As illustrated, the treatment assembly 1004 is again somewhat similar to the previous embodiments illustrated and described herein above. For example, in the embodiment illustrated in FIG. 10, the treatment assembly 1004 again includes an assembly shaft 1004A, and an energy director 1004B that is coupled to and/or secured to the assembly shaft 1004A. Additionally, in some embodiments, the treatment assembly 1004 and/or the energy director 1004B are movable between a retracted position (and collapsed state) and a deployed position (and expanded state). The deployed position and expanded state for the treatment assembly 1004 and/or the energy director 1004B is illustrated in FIG. 10. As shown, when the treatment assembly 1004 and/or the energy director 10046 are in the deployed position and the expanded state, a director distal end 1004D of the energy director 1004B can be positioned substantially adjacent to the vascular lesions 106A at the treatment site 106.

The assembly shaft 1004A and the energy director 1004B are somewhat similar to the embodiments illustrated and described herein above. For example, the assembly shaft 1004A can again include one or more inflation ports 1060 through which the catheter fluid 132 (illustrated in FIG. 1) can be directed into a director interior 1046 as defined by a director wall 1030 of the energy director 10046; and the assembly shaft 1004A can again define one or more energy guide lumens 1062 through which the one or more energy guides 1022A can extend. During use of the treatment assembly 1004, the energy director 1004B is again configured to receive and retain the catheter fluid 132 substantially within the director interior 1046 of the energy director 10046, which enables the creation of a plasma within the director interior 1046. The energy director 1004B is further configured to direct the energy from the plasma, such as in the form of one or more plasma bubbles 1034 and/or corresponding pressure waves generated near the guide distal end 1022D of the energy guide 1022A, toward the vascular lesions 106A at the treatment site 106.

However, in this embodiment, the energy director 10046 is substantially spherical-shaped, and includes a director proximal end 1004P that is coupled to the assembly shaft 1004A, and a director distal end 1004D that is positioned away from the assembly shaft 1004A. Additionally, the energy director 1004B further includes a director aperture 1068 (illustrated in phantom) that is formed into the director distal end 1004D of the energy director 1004B. With such design, the energy from the plasma formed in the catheter fluid 132 within the director interior 1046, such as in the form of the one or more plasma bubbles 1034 and/or corresponding pressure waves, can be more precisely and accurately directed toward the vascular lesions 106A on and/or adjacent to the leaflets 108B of the heart valve 108 at the treatment site 106. The director aperture 1068 can further help maintain a desired fluid pressure for the catheter fluid 132 within the director interior 1046, so as to further assist the energy director 1004B in maintaining its desired expanded state. It is appreciated that the director aperture 1068 can have any suitable size and shape for purposes of directing the plasma energy toward the vascular lesions 106A at the treatment site 106 as desired.

It should be noted that, as used in this specification and the appended claims, the singular forms “a,” “an,” and “the” include plural referents unless the content and/or context clearly dictates otherwise. It should also be noted that the term “or” is generally employed in its sense including “and/or” unless the content or context clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

The headings used herein are provided for consistency with suggestions under 37 CFR 1.77 or otherwise to provide organizational cues. These headings shall not be viewed to limit or characterize the invention(s) set out in any claims that may issue from this disclosure. As an example, a description of a technology in the “Background” is not an admission that technology is prior art to any invention(s) in this disclosure. Neither is the “Summary” or “Abstract” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

It is understood that although a number of different embodiments of the catheter systems have been illustrated and described herein, one or more features of any one embodiment can be combined with one or more features of one or more of the other embodiments, provided that such combination satisfies the intent of the present invention.

While a number of exemplary aspects and embodiments of the catheter systems have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope, and no limitations are intended to the details of construction or design herein shown.

What is claimed is:

1. A catheter system for treating a treatment site within or adjacent to a heart valve within a body of a patient, the catheter system comprising:
an energy source that generates energy;
an energy guide including a guide proximal end and a guide distal end, the energy guide being configured to receive energy from the energy source and guide the energy from the guide proximal end toward the guide distal end; and
an energy director including a director wall that defines a director interior, and a director distal end that is selectively positioned substantially adjacent to the treatment site, the director distal end being at least partially open in a direction toward the treatment site;
wherein the guide distal end of the energy guide is positioned within the director interior.

2. The catheter system of claim 1 wherein the energy director is substantially cone-shaped, the energy director further including a director proximal end that is smaller than the director distal end.

3. The catheter system of claim 1 wherein the director distal end is fully open in a direction toward the treatment site.

4. The catheter system of claim 1 wherein the guide distal end is partially closed in a direction toward the treatment site, and the guide distal end including a director aperture that is open in a direction toward the treatment site.

5. The catheter system of claim 1 wherein the energy director is somewhat spherical-shaped.

6. The catheter system of claim 5 wherein the guide distal end is partially closed and includes a director aperture that is open in a direction toward the treatment site.

7. The catheter system of claim 1 wherein the energy director is configured to receive a catheter fluid within the director interior; and wherein the energy guide guides the energy into the catheter fluid within the director interior so that plasma is formed in the catheter fluid within the director interior.

8. The catheter system of claim 7 wherein the plasma formation causes rapid bubble formation and generates one or more pressure waves within the catheter fluid that impart a force upon the treatment site.

9. The catheter system of claim 1 further comprising a second energy guide including a second guide proximal end and a second guide distal end, the second energy guide being configured to receive energy from the energy source and guide the energy from the second guide proximal end toward the second guide distal end, the second guide distal end of the second energy guide being positioned within the director interior.

10. The catheter system of claim 1 further comprising an assembly shaft, wherein the energy director is coupled to the assembly shaft.

11. The catheter system of claim 10 wherein the assembly shaft includes an inflation port, and a catheter fluid is directed into the director interior of the energy director through the inflation port.

12. The catheter system of claim 10 wherein the assembly shaft includes an energy guide lumen; and wherein at least a portion of the energy guide extends through the energy guide lumen.

13. The catheter system of claim 10 wherein the assembly shaft is substantially cylindrical-shaped.

14. The catheter system of claim 1 wherein the energy director is selectively movable between a retracted position and a deployed position so that when the energy director is in the retracted position, the energy director is positioned substantially within an outer sheath, and when the energy director is in the deployed position, the energy director is positioned outside of and extends away from the outer sheath.

15. The catheter system of claim 14 wherein when the energy director is in the deployed position, the director distal end is positioned substantially adjacent to the treatment site.

16. The catheter system of claim 14 wherein when the energy director is in the retracted position, the energy director is configured to be in a collapsed state.

17. The catheter system of claim 16 wherein when the energy director is in the deployed position, the energy director is configured to move from the collapsed state to an expanded state.

18. The catheter system of claim 17 further comprising an expansion assistance structure that is coupled to the director wall, the expansion assistance structure being configured to assist the energy director to move from the collapsed state to the expanded state.

19. The catheter system of claim 1 wherein the heart valve includes one or more leaflets, the catheter system further including a leaflet support assembly including (i) a support shaft that is configured to extend through the heart valve, and (ii) a leaflet supporter that is coupled to the support shaft and extends substantially perpendicularly away from the support shaft, the director distal end of the energy director and the leaflet supporter being selectively positionable on opposite sides of one of the leaflets.

20. The catheter system of claim 1 wherein the energy director is formed from one or more of silicone, plastic, polyester, spun polytetrafluoroethylene and nylon.

21. The catheter system of any one of claims 1-30 wherein the energy source is a laser that provides pulses of laser energy, and the energy guide includes an optical fiber.

22. The catheter system of claim 1 wherein the energy source is a high voltage energy source that provides pulses of high voltage.

23. The catheter system of claim 22 wherein the energy guide includes an electrode pair including spaced apart electrodes that extend into the director interior; and wherein pulses of high voltage from the energy source are applied to the electrodes and form an electrical arc across the electrodes.

24. A method for treating a treatment site within or adjacent to a heart valve within a body of a patient, the method including the step of utilizing the catheter system of claim 1.
